(19) United States
(12) Patent Application Publication
(10) Pub. No.: US 2023/0238136 A1
(43) Pub. Date: Jul. 27, 2023

(54) ASSESSMENT OF PROBABILITY OF BONE FRACTURE

(71) Applicants: Zohar YOSIBASH, Lehavim (IL); Nir TRABELSI, Beer Sheva (IL); Charles MILGROM, Moshav Aminadav (IL); Kent MYERS, Meredith, NH (US)

(72) Inventors: Zohar YOSIBASH, Lehavim (IL); Nir TRABELSI, Beer Sheva (IL)

(21) Appl. No.: 18/010,691

(22) PCT Filed: Jun. 17, 2021
(86) PCT No.: PCT/IL2021/050742

§ 371 Date: Dec. 15, 2022

Related U.S. Application Data
(60) Provisional application No. 63/040,549, filed on Jun. 18, 2020.

(51) Int. Cl. G16H 50/20; G06T 7/73; G06T 7/00; G16H 30/20

(52) U.S. Cl. CPC G16H 50/20; G06T 7/73; G06T 7/0012; G16H 30/20; G06T 2207/30008; G06T 2200/04; G06T 2207/20081; G06T 2207/20076

(57) ABSTRACT

A patient-specific assessment of fracture probability for the femur proximal end is provided. 3D locations of the femur head center, a point on the femoral shaft center, and the femoral intercondylar notch are determined from a clinical image. A frontal plane, a perpendicular thereunto and a bone shaft axis are determined from the 3D locations. An FEA coordinate system is defined from the frontal plane, the perpendicular and the axis. Two FEA analyses are performed, one for neck fracture and one for pertrochanteric fracture, with the same displacement constraints and the same load magnitude but different load angles. The femur proximal end is divided into four anatomically-based regions. For each region and each load, maximum tensile and compressive principal strains are determined and, based on the body weight and the principal strains, a likelihood of fracture is obtained. The minimum of these 8 likelihoods gives the probability of fracture.

### FIELD OF THE INVENTION

The present invention generally pertains to a system and method for providing a patient-specific assessment of the probability of fracture of the proximal end of a femur.

### BACKGROUND OF THE INVENTION

Osteoporosis and Diabetes mellitus (DM) impair bone strength, and are prevalent risk factors for hip fracture when falling on the side, but currently there is no reliable tool to assess the risk of hip fracture for either population. The reason is that most risk stratifying methods rely on bone mineral density, which, at least for diabetic patients, impairs bone strength by other mechanisms, rendering current tests ineffective.

Hip fractures in the elderly are considered the direst complication of osteoporosis/low bone mass, and account for most of the health and financial burden of this condition¹. The incidence of hip fracture ranges between 200-400 per 100,000 for most developed countries, and it rises with increasing age. At the same time, the prevalence of diabetes mellitus (DM), and especially Type 2 diabetes (T2DM), is also increasing rapidly³. Among its numerous complications, diabetes mellitus is a risk factor for sustaining a hip fracture⁴: Diabetic adults have a twofold greater risk of hip fractures (RR 2.07; 95% CI 1.83-2.33), although this is more pronounced in type 1 diabetes (RR 5.76; 95% CI 3.66-9.07) than that in type 2 diabetes (RR 1.34; 95% CI 1.19-1.51)⁵. With improving survival of T2DM patients and rising life expectancy in general, it is estimated that the prevalence of hip fractures among T2DM patients will continue to increase.

The most popular method to assess the risk for hip fracture is by calculating bone mineral density (BMD) by means of dual-energy X-ray absorptiometry (DXA). BMD is a good surrogate for assessing fracture risk in the general population, as the risk for fracture increases by a factor of 1.5 to 3.0 for each decrease of one unit in the T-score scale⁶. However, this method does not seem to work for T2DM patients, who display a “diabetic paradox”—an increased risk for fragility fractures despite normal bone mineral density. Other widely used fracture risk index—the FRAX—was also found to underestimates the risk of major osteoporotic fractures in T2DM⁸, necessitating the use of several adjustments in T2DM patients^{9, 10}. The use of trabecular bone score (TBS), an indirect index of trabecular architecture that can be derived from a spine DXA image, has recently shown promise in stratifying fracture risk in T2DM patients, but this was assessed only for vertebral fracture risk^{9, 11}. Despite continuing research and improvement, assessing fracture risk in T2DM patients remains challenging¹⁰.

In recent years methods based on finite element analysis models generated from computed tomography scans (CTFEA) have been suggested as a means to assess bone strength and predict fracture risk¹². In a systematic review including 12 in vitro studies, CTFEA was found more accurate in predicting fracture risk than commonly used fracture risk assessment techniques¹³.

U.S. Pat. No. 9,245,069 discloses methods for modeling structural behavior of a bone. For example, the methods can model the structural behavior of a femur, and in particular, a proximal femur, under conditions representing a fall onto the greater trochanter of the proximal femur. In such an embodiment, the method can comprise determining a strength of the femur, by a processor and based on (a) the elastic modulus, (b) the cortical bone yield strength, (c) the stress level σₘᵢₙ, and (d) the post-yield stress-strain slope of the proximal femur.

However, the main concern of U.S. Pat. No. 9,245,069 is the determination of stress-strain curves for the bone elements. The constraints applied to the bone during the simulation are not explicitly stated, although displacement constraints appear to be used, and the probability of fracture is determined from the presence of strains greater than the bone failure strain at some location in the simulated bone.

The published article by Altai et al.¹⁴ discloses the effect of different side fall boundary and loading conditions on a retrospective cohort of 98 postmenopausal women to test models' ability to discriminate fracture and control cases for fracture of the proximal portion of the femur (head and neck of the femur). Three different boundary conditions (Linear, Multi-point constraints (MPC) and Contact model) were investigated under various anterolateral and posterolateral falls. The system and method tested therein determines bone properties automatically from bone images, applies constraints to the bones at predetermined angles, determines the maximum principal strains for a predetermined area of the bone, and discriminates between bones likely to fracture and bones unlikely to fracture based on an averaged maximum principal strain.

However, the constraints applied, both at the distal end of the simulated bone and at the contact point on the lateral side of the greater trochanter (a simulation of the contact point between the femur and a hard surface) differ significantly from those of the present invention. The averaging is performed over the entire neck region of the simulated bone, and area under the receiver operating characteristic curve was used to determine a hip strength score. Also, there is no unique algorithm to apply the load in the same anatomical conditions to different patients based on anatomical points, the methods do not take into consideration body weight, nor do they or an average of a plurality of specific boundary conditions to determine fracture.

It is therefore a long felt need to provide a system and method for determining the probability of fracture of the proximal femur that does not depend on area under a curve, that does not depend on either a rigid constraint or an MPC constraint at the distal end of the simulated bone, where the orientation of the simulated bone has no arbitrary components.

### SUMMARY OF THE INVENTION

It is an object of the present invention to disclose a system and method for providing a patient-specific assessment the probability of fracture of the proximal end of a femur

It is another object of the present invention to disclose a method for determining at least three anatomical points of a femur from at least one clinical image comprising steps of:
segmenting said at least one clinical image to obtain a 3D image of said femur;
applying a location-determining algorithm selected from a group consisting of K Nearest Neighbor (KNN), Support Vector Machine (SVM) learning, Decision Tree Learning, and any combination thereof to determine a 3D location of an intercondylar notch for said 3D image;
determining a 3D location of a femur head center (HC) and a point along a shaft center (SC); thereby determining said at least three anatomical points.

It is another object of the present invention to disclose the method as disclosed above, additionally comprising training said location-determining algorithm by steps of:
obtaining, for at least one femur, at least one scan of said femur, said scan including all of said at least three anatomical points;
determining, for at least one of said at least three anatomical points, a location, each said location being a determined anatomical point;
removing a portion from said 3D image to generate a training image, said removed portion comprising said at least one of said at least three anatomical points;
training said KNN by, for each of said at least one femurs, said KNN executing steps of:
   determining, for at least one of said at least three anatomical points, a location, each said location being a training anatomical point;
   generating, by comparing each of said training anatomical points and a corresponding determined anatomical point, an anatomical point difference factor;
   comparing each said anatomical point difference factor with a range of a predetermined difference factor;
   said anatomical point difference factor being outside said range of said predetermined difference factor, modifying said KNN; and
   repeating said steps of determining, generating, comparing and modifying until each of said anatomical point difference factors being inside said range of said predetermined difference factor.

It is another object of the present invention to disclose the method as disclosed above, wherein said range of said predetermined difference factor is different for at least two of said at least three anatomical points.

It is another object of the present invention to disclose the method as disclosed above, wherein said at least one of said at least three anatomical points is an intercondylar point.

It is another object of the present invention to disclose the method as disclosed above, wherein said training image comprises a proximal portion of the femur.

It is another object of the present invention to disclose the method as disclosed above, wherein said removed portion comprises said intercondylar point.

It is another object of the present invention to disclose the method as disclosed above, wherein said at least three anatomical points comprises a center of a femoral head; a center of a femoral shaft section a predetermined distance distal to the lesser trochanter; and a femoral intercondylar notch.

It is another object of the present invention to disclose the method as disclosed above, wherein said predetermined distance distal to the lesser trochanter is in a range from 10 mm to 50 mm distal to the lesser trochanter.

It is another object of the present invention to disclose the method as disclosed above, wherein said predetermined distance distal to the lesser trochanter is 20 mm distal to the lesser trochanter.

It is another object of the present invention to disclose the method as disclosed above, wherein a method of determining the 3D location of the HC comprises steps of:
identifying a most proximal point on the femur head;
identifying a most distal point on the femur head;
identifying a middle section of the femur head, said middle section being a section halfway between said most proximal point and said most distal point, a Z coordinate of said HC being a Z coordinate of said middle section;
selecting an HC section, said HC section being 10 mm distal to the most proximal point; and
determining a center of said HC section, an X coordinate of said HC being an X coordinate of said HC section and a Y coordinate of said HC being a Y coordinate of said HC section.

It is another object of the present invention to disclose the method as disclosed above, wherein a method of determining the 3D location of the SC comprises steps of:
identifying a location of a lesser trochanter in said scan;
selecting an SC section, said SC section being a predetermined distance distal to said lesser trochanter, a Z coordinate of said SC being a Z coordinate of said SC section; and
determining a center of said SC section, an X coordinate of said SC being an X coordinate of said SC section and a Y coordinate of said SC being a Y coordinate of said SC section.

It is another object of the present invention to disclose the method as disclosed above, wherein said predetermined distance distal to said lesser trochanter is in a range from 10 mm to 30 mm

It is another object of the present invention to disclose the method as disclosed above, wherein said predetermined distance distal to said lesser trochanter is 20 mm

It is another object of the present invention to disclose a method of automatically generating bone-specific constraints for an FEA model of a femur in an FEA coordinate system, comprising steps of:
defining a frontal plane in said FEA coordinate system from three predetermined anatomic points of said femur;
defining a perpendicular to said frontal plane;
defining a bone shaft axis from a predetermined two of said three predetermined anatomic points;
generating an angle γ, said angle γ being an angle between a force vector and said shaft axis;
generating an angle δ, the angle δ being an angle between said force vector and said perpendicular to said frontal plane;
applying a force vector with magnitude equal to a predetermined force value at said angles γ and δ;
applying zero displacement in a direction parallel to said force vector on a greater trochanter lateral end;
applying, at a distal end of said FEA model of said femur, a zero displacement in a direction parallel to said bone shaft axis and a zero displacement in a direction parallel to said perpendicular; and
applying, at a distal end of said FEA model of said femur, zero force in a direction perpendicular to both said shaft axis and said perpendicular.

It is another object of the present invention to disclose the method as disclosed above, wherein at least two sets of said bone specific constraints are generated, one of said at least two sets having differing from at least one other of said at least two sets in a member of a group consisting of angle γ, angle δ and any combination thereof.

It is another object of the present invention to disclose the method as disclosed above, wherein said at least two sets of said bone specific constraints comprises a set with angle γ in a range of 6° to 14° and angle δ in a range of 11° to 19°.

It is another object of the present invention to disclose the method as disclosed above, wherein said at least two sets of said bone specific constraints comprises a set with angle γ in a range of 26° to 34° and angle δ in a range of 41° to 49°.

It is another object of the present invention to disclose the method as disclosed above, wherein said at least two sets of said bone specific constraints comprises a set with angle γ being 10° and angle δ being 15°.

It is another object of the present invention to disclose the method as disclosed above, wherein said at least two sets of said bone specific constraints comprises a set with angle γ being 30° and angle δ being 45°.

It is another object of the present invention to disclose the method as disclosed above, wherein said predetermined force value is equal to a body mass of a patient with said femur.

It is another object of the present invention to disclose the method as disclosed above, wherein said three anatomical points are a center of a femoral head; a center of a femoral shaft section a predetermined distance distal to a lesser trochanter; and a femoral intercondylar notch.

It is another object of the present invention to disclose the method as disclosed above, wherein said predetermined distance distal to said lesser trochanter is in a range from 10 mm to 50 mm distal to the lesser trochanter.

It is another object of the present invention to disclose the method as disclosed above, wherein said predetermined distance distal to said lesser trochanter is 20 mm distal to the lesser trochanter.

It is another object of the present invention to disclose a method of determining average strain in a femur from an FEA analysis of a femur under a predetermined load, comprising steps of:
automatically dividing a proximal end of said femur into four regions determined by anatomical points, said anatomical points being anterior and posterior superior neck, anterior and posterior inferior neck; greater trochanter and posterior lesser trochanter and anterior lesser trochanter; for each point on an exterior surface of said femur, averaging a principal compressive strain and a principal tensile strain over a predetermined area, generating an averaged principal compressive strain and an averaged principal tensile strain;
for each of said four regions, determining a maximum principal compressive strain, said maximum principal compressive strain being said averaged principal compressive strain having a maximum absolute value of said nodal averaged principal compressive strain of said area in said region; and
for each of said four regions, determining a maximum principal tensile strain, said maximum principal tensile strain being a maximum value of said averaged principal tensile strain of said region.

It is another object of the present invention to disclose the method as disclosed above, wherein said four regions are a superior neck, an inferior neck, an anterior trochanter and a posterior trochanter.

It is another object of the present invention to disclose the method as disclosed above, wherein said predetermined area is in a range from 2.5 mm² to 10 mm²

It is another object of the present invention to disclose the method as disclosed above, wherein said predetermined area is 5 mm²

It is another object of the present invention to disclose a method of determining likelihood of fracture of a femur, comprising steps of:
executing at least two FEA analyses, said FEA analyses having constraint sets Fall_{N} and Fall_{P};
extracting a maximum tensile principal strain E1ₘₐₓ for said femur;
extracting a maximum compressive principal strain E3ₘᵢₙ for said femur;
for each of said four regions and for each of said constraint sets, calculating a body weight factor in tension (BWFₜₑₙ) for each said region from

$$B W {F}_{ten} = \frac{0.0073}{BW \cdot E {1}_{max}}$$

where BW is the body mass of a patient with said femur and E1ₘₐₓ is said maximum tensile principal strain;
for each of said four regions and for each of said constraint sets, calculating a body weight factor in compression (BWF_{com}) for each said region from

$$B W {F}_{com} = - \frac{0.0 \; 086}{BW \cdot E {3}_{min}}$$

where BW is the body mass of a patient with said femur and E3ₘᵢₙ is said maximum compressive principal strain;
for each of said four regions, calculating a hip strength score for each region (HSS_{R}) from

$$H S {S}_{R} = \frac{1}{2} ( B W {F}_{com , FallN} + B W {F}_{com , FallP} )$$

where BWF_{com,fallN} for each region is the body weight factor in compression for that region under the constraint set Fall_{N} and where BWF_{com,fallP} for each region is the body weight factor in compression for that region under the constraint set Fall_{P};
calculating a hip strength score HSS for a femur as a minimum of the four values HSS_{R};
wherein said femur is likely to fracture if said HSS is less than a predetermined HSS value.

It is another object of the present invention to disclose the method as disclosed above, wherein said constraint sets Fall_{N} and Fall_{P} have constraints comprising:
applying a force vector with magnitude equal to a predetermined force value at angles γ and δ;
applying zero displacement in a direction parallel to said force vector on a greater trochanter lateral end;
applying, at a distal end of said FEA model of said femur, a zero displacement in a direction parallel to a bone shaft axis and a zero displacement in a direction parallel to a perpendicular to a bone frontal plane;
applying, at a distal end of said FEA model of said femur, zero force in a direction perpendicular to both said bone shaft axis and said perpendicular to a bone frontal plane.

It is another object of the present invention to disclose the method as disclosed above, wherein said constraint set Fall_{N} comprises a set with angle γ in a range of 6° to 14° and angle δ in a range of 11° to 19°.

It is another object of the present invention to disclose the method as disclosed above, wherein said constraint set Fall_{P} comprises a set with angle γ in a range of 26° to 34° and angle δ in a range of 41° to 49°.

It is another object of the present invention to disclose the method as disclosed above, wherein said constraint set Fall_{N} comprises a set with angle γ being 10° and angle δ being 15°.

It is another object of the present invention to disclose the method as disclosed above, wherein said constraint set Fall_{P} comprises a set with angle γ being 30° and angle δ being 45°.

It is another object of the present invention to disclose the method as disclosed above, wherein said predetermined force value is equal to a body mass of a patient with said femur.

It is another object of the present invention to disclose the method as disclosed above, wherein said predetermined HSS value is in a range from 1.5 to 4.5.

It is another object of the present invention to disclose the method as disclosed above, wherein, for T2DM patients, said predetermined HSS value is in a range from 2.0 to 2.3.

It is another object of the present invention to disclose the method as disclosed above, wherein, for T2DM patients, said predetermined HSS value is 2.2.

It is another object of the present invention to disclose a system for determining at least three anatomical points of a femur from at least one clinical image comprising software configured, when executed, to:
segment said at least one clinical image to obtain a 3D image of said femur;
apply a location-determining algorithm selected from a group consisting of K Nearest Neighbor (KNN), Support Vector Machine (SVM) learning, Decision Tree Learning, and any combination thereof to determine a 3D location of an intercondylar notch for said 3D image;
determine a 3D location of a femur head center (HC) and a point along a shaft center (SC); wherein said software is configured, when executed, to determine said at least three anatomical points.

It is another object of the present invention to disclose the system as disclosed above, additionally comprising software configured, when executed, to train said location-determining algorithm by:
obtaining, for at least one femur, at least one scan of said femur, said scan including all of said at least three anatomical points;
determining, for at least one of said at least three anatomical points, a location, each said location being a determined anatomical point;
removing a portion from said 3D image to generate a training image, said removed portion comprising said at least one of said at least three anatomical points;
training said KNN by, for each of said at least one femurs, said KNN executing steps of:
   determining, for at least one of said at least three anatomical points, a location, each said location being a training anatomical point;
   generating, by comparing each of said training anatomical points and a corresponding determined anatomical point, an anatomical point difference factor;
   comparing each said anatomical point difference factor with a range of a predetermined difference factor;
   said anatomical point difference factor being outside said range of said predetermined difference factor, modifying said KNN; and
   repeating said steps of determining, generating, comparing and modifying until each of said anatomical point difference factors being inside said range of said predetermined difference factor.

It is another object of the present invention to disclose the system as disclosed above, wherein said range of said predetermined difference factor is different for at least two of said at least three anatomical points.

It is another object of the present invention to disclose the system as disclosed above, wherein said at least one of said at least three anatomical points is an intercondylar point.

It is another object of the present invention to disclose the system as disclosed above, wherein said training image comprises a proximal portion of the femur.

It is another object of the present invention to disclose the system as disclosed above, wherein said removed portion comprises said intercondylar point.

It is another object of the present invention to disclose the system as disclosed above, wherein said at least three anatomical points comprises a center of a femoral head; a center of a femoral shaft section a predetermined distance distal to the lesser trochanter; and a femoral intercondylar notch.

It is another object of the present invention to disclose the system as disclosed above, wherein said predetermined distance distal to the lesser trochanter is in a range from 10 mm to 50 mm distal to the lesser trochanter.

It is another object of the present invention to disclose the system as disclosed above, wherein said predetermined distance distal to the lesser trochanter is 20 mm distal to the lesser trochanter.

It is another object of the present invention to disclose the system as disclosed above, wherein said software is additionally configured, when executed, to determine the 3D location of the HC by:
identifying a most proximal point on the femur head;
identifying a most distal point on the femur head;
identifying a middle section of the femur head, said middle section being a section halfway between said most proximal point and said most distal point, a Z coordinate of said HC being a Z coordinate of said middle section;
selecting an HC section, said HC section being 10 mm distal to the most proximal point; and
determining a center of said HC section, an X coordinate of said HC being an X coordinate of said HC section and a Y coordinate of said HC being a Y coordinate of said HC section.

It is another object of the present invention to disclose the system as disclosed above, wherein said software is additionally configured, when executed, to determine the 3D location of the SC by:
identifying a location of a lesser trochanter in said scan;
selecting an SC section, said SC section being a predetermined distance distal to said lesser trochanter, a Z coordinate of said SC being a Z coordinate of said SC section; and
determining a center of said SC section, an X coordinate of said SC being an X coordinate of
said SC section and a Y coordinate of said SC being a Y coordinate of said SC section.

It is another object of the present invention to disclose the system as disclosed above, wherein said predetermined distance distal to said lesser trochanter is in a range from 10 mm to 30 mm

It is another object of the present invention to disclose the system as disclosed above, wherein said predetermined distance distal to said lesser trochanter is 20 mm

It is another object of the present invention to disclose a system of automatically generating bone-specific constraints for an FEA model of a femur in an FEA coordinate system, comprising software configured, when executed, to:
define a frontal plane in said FEA coordinate system from three predetermined anatomic points of said femur;
define a perpendicular to said frontal plane;
define a bone shaft axis from a predetermined two of said three predetermined anatomic points;

generate an angle γ, said angle γ being an angle between a force vector and said shaft axis;

generate an angle δ, the angle δ being an angle between said force vector and said perpendicular to said frontal plane;

apply a force vector with magnitude equal to a predetermined force value at said angles γ and δ;

apply zero displacement in a direction parallel to said force vector on a greater trochanter lateral end;

apply, at a distal end of said FEA model of said femur, a zero displacement in a direction parallel to said bone shaft axis and a zero displacement in a direction parallel to said perpendicular; and
apply, at a distal end of said FEA model of said femur, zero force in a direction perpendicular to both said shaft axis and said perpendicular.

It is another object of the present invention to disclose the system as disclosed above, wherein at least two sets of said bone specific constraints are generated, one of said at least two sets having differing from at least one other of said at least two sets in a member of a group consisting of angle γ, angle δ and any combination thereof.

It is another object of the present invention to disclose the system as disclosed above, wherein said at least two sets of said bone specific constraints comprises a set with angle γ in a range of 6° to 14° and angle δ in a range of 11° to 19°.

It is another object of the present invention to disclose the system as disclosed above, wherein said at least two sets of said bone specific constraints comprises a set with angle γ in a range of 26° to 34° and angle δ in a range of 41° to 49°.

It is another object of the present invention to disclose the system as disclosed above, wherein said at least two sets of said bone specific constraints comprises a set with angle γ being 10° and angle δ being 15°.

It is another object of the present invention to disclose the system as disclosed above, wherein said at least two sets of said bone specific constraints comprises a set with angle γ being 30° and angle δ being 45°.

It is another object of the present invention to disclose the system as disclosed above, wherein said predetermined force value is equal to a body mass of a patient with said femur.

It is another object of the present invention to disclose the system as disclosed above, wherein said three anatomical points are a center of a femoral head; a center of a femoral shaft section a predetermined distance distal to a lesser trochanter; and a femoral intercondylar notch.

It is another object of the present invention to disclose the system as disclosed above, wherein said predetermined distance distal to said lesser trochanter is in a range from 10 mm to 50 mm distal to the lesser trochanter.

It is another object of the present invention to disclose the system as disclosed above, wherein said predetermined distance distal to said lesser trochanter is 20 mm distal to the lesser trochanter.

It is another object of the present invention to disclose a system of determining average strain in a femur from an FEA analysis of a femur under a predetermined load, comprising software configured, when executed, to:
automatically divide a proximal end of said femur into four regions determined by anatomical points, said anatomical points being anterior and posterior superior neck, anterior and posterior inferior neck; greater trochanter and posterior lesser trochanter and anterior lesser trochanter; for each point on an exterior surface of said femur, averaging a principal compressive strain and a principal tensile strain over a predetermined area, generating an averaged principal compressive strain and an averaged principal tensile strain;
for each of said four regions, determine a maximum principal compressive strain, said maximum principal compressive strain being said averaged principal compressive strain having a maximum absolute value of said nodal averaged principal compressive strain of said area in said region; and
for each of said four regions, determine a maximum principal tensile strain, said maximum principal tensile strain being a maximum value of said averaged principal tensile strain of said region.

It is another object of the present invention to disclose the system as disclosed above, wherein said four regions are a superior neck, an inferior neck, an anterior trochanter and a posterior trochanter.

It is another object of the present invention to disclose the system as disclosed above, wherein said predetermined area is in a range from 2.5 mm² to 10 mm²

It is another object of the present invention to disclose the system as disclosed above, wherein said predetermined area is 5 mm²

It is another object of the present invention to disclose a system of determining likelihood of fracture of a femur, comprising software configured, when executed, to:
execute at least two FEA analyses, said FEA analyses having constraint sets Fall_{N} and Fall_{P};
extract a maximum tensile principal strain E1ₘₐₓ for said femur;
extract a maximum compressive principal strain E3ₘᵢₙ for said femur;
for each of said four regions and for each of said constraint sets, calculate a body weight factor in tension (BWFₜₑₙ) for each said region from

$$B W {F}_{ten} = \frac{0.0073}{BW \cdot E {1}_{max}}$$

where BW is the body mass of a patient with said femur and E1ₘₐₓ is said maximum tensile principal strain;
for each of said four regions and for each of said constraint sets, calculate a body weight factor in compression (BWF_{com}) for each said region from

$$B W {F}_{com} = - \frac{0.0 \; 086}{BW \cdot E {3}_{min}}$$

where BW is the body mass of a patient with said femur and E3ₘᵢₙ is said maximum compressive principal strain;
for each of said four regions, calculate a hip strength score for each region (HSS_{R}) from

$$H S {S}_{R} = \frac{1}{2} ( B W {F}_{com , FallN} + B W {F}_{com , FallP} )$$

where BWF_{com, fallN} for each region is the body weight factor in compression for that region under the constraint set Fall_{N} and where BWF_{com,fallP} for each region is the body weight factor in compression for that region under the constraint set Fall_{P};
calculate a hip strength score HSS for a femur as a minimum of the four values HSS_{R};
wherein said femur is likely to fracture if said HSS is less than a predetermined HSS value.

It is another object of the present invention to disclose the system as disclosed above, wherein said constraint sets Fall_{N} and Fall_{P} have constraints comprising:
applying a force vector with magnitude equal to a predetermined force value at angles γ and δ;
applying zero displacement in a direction parallel to said force vector on a greater trochanter lateral end;
applying, at a distal end of said FEA model of said femur, a zero displacement in a direction parallel to a bone shaft axis and a zero displacement in a direction parallel to a perpendicular to a bone frontal plane;
applying, at a distal end of said FEA model of said femur, zero force in a direction perpendicular to both said bone shaft axis and said perpendicular to a bone frontal plane.

It is another object of the present invention to disclose the system as disclosed above, wherein said constraint set Fall_{N} comprises a set with angle γ in a range of 6° to 14° and angle δ in a range of 11° to 19°.

It is another object of the present invention to disclose the system as disclosed above, wherein said constraint set Fall_{P} comprises a set with angle γ in a range of 26° to 34° and angle δ in a range of 41° to 49°.

It is another object of the present invention to disclose the system as disclosed above, wherein said constraint set Fall_{N} comprises a set with angle γ being 10° and angle δ being 15°.

It is another object of the present invention to disclose the system as disclosed above, wherein said constraint set Fall_{P} comprises a set with angle γ being 30° and angle δ being 45°.

It is another object of the present invention to disclose the system as disclosed above, wherein said predetermined force value is equal to a body mass of a patient with said femur.

It is another object of the present invention to disclose the system as disclosed above, wherein said predetermined HSS value is in a range from 1.5 to 4.5.

It is another object of the present invention to disclose the system as disclosed above, wherein, for T2DM patients, said predetermined HSS value is in a range from 2.0 to 2.3.

It is another object of the present invention to disclose the system as disclosed above, wherein, for T2DM patients, said predetermined HSS value is 2.2.

### BRIEF DESCRIPTION OF THE FIGURES

In order to better understand the invention and its implementation in practice, a plurality of embodiments will now be described, by way of non-limiting example only, with reference to the accompanying drawings, wherein

FIG. 1 schematically illustrates an algorithm for generating a patient-specific FE model of a femur;

FIG. 2 schematically illustrates an algorithm for the determination of the coordinates of the femoral head center (HC) in a CT scan;

FIG. 3A-D schematically illustrates an algorithm for the determination of the coordinates of the midshaft point 20 mm below the lesser trochanter;

FIG. 4 shows an example of the errors in the angles γ and δ (degrees) resulting from estimation of the intercondylar notch by KNN, as a function of the length of the proximal femur in the CT scan;

FIG. 5A-D depicts precise boundary conditions based on anatomical points for a sideways fall configuration;

FIG. 6 illustrates the femoral regions of interest, the superior neck, the inferior neck, the anterior trochanter and the posterior trochanter;

FIG. 7A illustrates neck fracture; FIG. 7B illustrates the strains resulting from neck fracture loading (Fall_{N}); FIG. 7C illustrates the strains resulting from pertrochanteric fracture loading (Fall_{P}); and FIG. 7D illustrates pertrochanteric fracture;

FIG. 8A-B illustrates the procedure for selecting a study group and a control group to determine the threshold HSS;

FIG. 9A-D illustrates receiver operating characteristic (ROC) curves for the multivariate logistic regression model using four different cut-off points for the combined score; and

FIG. 10 shows the HSS as a function of age.

### DETAILED DESCRIPTION OF THE PREFERRED EMBODIMENTS

The following description is provided, alongside all chapters of the present invention, so as to enable any person skilled in the art to make use of said invention and sets forth the best modes contemplated by the inventor of carrying out this invention. Various modifications, however, will remain apparent to those skilled in the art, since the generic principles of the present invention have been defined specifically to provide a means and method for providing a patient-specific assessment the probability of fracture of the proximal end of a femur.

The term ‘about’ hereinafter refers to ±10% of a value.

The term ‘proximal’ hereinafter refers to a location closer to the head end of a femur.

The term ‘distal’ hereinafter refers to a location further from the head end of the femur and closer to the condylar end of a femur.

FEA analysis can be performed on a CT or other scan of a bone such as the proximal part of the femur to generate a patient-specific measure of hip bone strength termed hip strength score (HSS), where the HSS can clearly distinguish between a proximal femur likely to fracture and a proximal femur unlikely to fracture, thus enabling assessment of hip fracture risk. This HSS is a function of the body weight of the patient, to allow more accurate assessment of patients of different weights. As shown below, in an exemplary study, a significant difference (p=0.002) was found between the hip strength score (HSS) of T2DM patients who broke their hip in the year subsequent to a hip CT scan and T2DM patients who did not fracture their hip. This demonstrates that FEA using bone properties determinable from a CT or other scan can be used to assess hip fracture risk in the diabetic or osteopenic population, which currently lacks reliable patient-specific methods for assessing bone strength and fracture risk.

In recent years, a large number of studies showed the feasibility of using markers derived from existing CT scans to predict osteoporotic fractures¹⁵, and specifically hip fractures^{12, 16}. In the largest study thus far, Dagan et al.¹⁷ used CT derived markers (Vertebral compression fractures, lumbar trabecular density and simulated DXA T-score) on a population of 48,227 people aged 50-90 to assess the 5-year risk of osteoporotic fractures. Compared with the FRAX method without bone mineral density input, the CT based markers generated better predictions for any osteoporotic fracture (AUC of 70.9% Vs 69.1%), and non-inferior predictions for hip fracture (AUC 76.0% vs 75.1%). That study demonstrated the potential for using existing CT scans as a tool for assessing risk for osteoporotic fracture; however, the imaging biomarkers used in the CT analysis of that study, as in most previous studies, rely mostly on bone mineral density assessment. Thus, they are probably susceptible to the same “diabetic paradox” as the DXA test in diabetic patients.

FEA analysis generates markers of bone strength (material properties), and not bone mineral density. Measures of femoral strength have been shown to better predict hip fracture then hip BMD^{18, 19}. Only one previous study has demonstrated the use of femoral strength measurement derived from existing CT scans to predict hip fracture risk, the Fracture, Osteoporosis, and CT Utilization study (FOCUS). In that study, 1,959 patients aged 65 or older who sustained a hip fracture and who had a prior pelvic or abdominal CT scan and a DXA test, were compared to a sex matched group. Compared with DXA hip/spine t-score, the sensitivity for predicting hip fracture using the CT derived biomechanical markers was higher (women: 0.66 versus 0.59; men: 0.56 versus 0.48 after an average 3.7 years), with comparable respective specificity. The study population included 30% diabetic patients, but there is no sub analysis to determine the validity of this method specifically in those patients.

The CT utilization rate of 89% is on par with other published studies using opportunistic screening tools: Dagan et al reported 83.6% utilization rate¹⁷ and Adams et al reported 86% utilization rate²⁰, both in a very large and diverse populations.

CTFEA models, whether based on CT scans, X-rays, MRI scans, ultrasound or other scans capable of providing a measure of bone density as a function of position in the scan, use patient specific geometrical structure and spatial distribution of material properties, not just bone mineral density, to assess bone strength, thereby bypassing the “diabetic paradox”, able to assess accurately hip fracture risk in T2DM patients²⁰. CTFEA derived measures of bone strength have been proven to predict hip fracture independent of hip BMD in the general population²¹. Herein, the weight of the patient is also considered as one of the parameters in determining the load applied to the CTFEA model of the patient's femur.

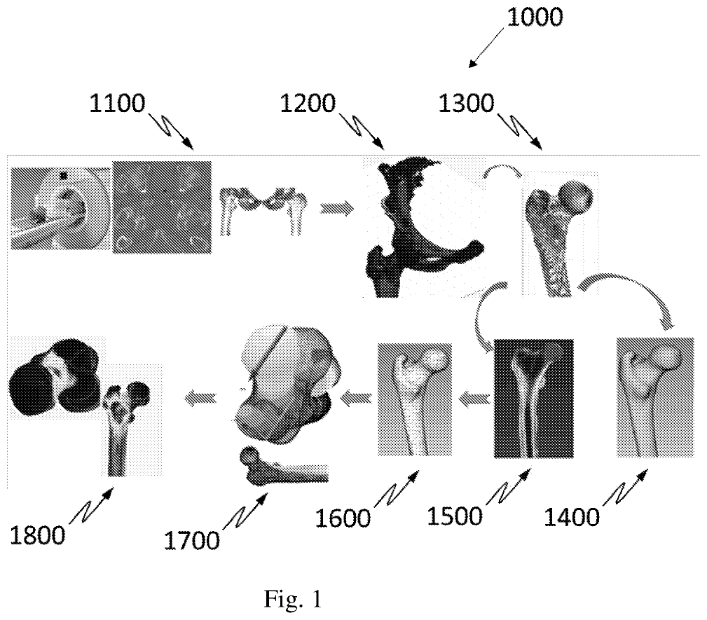

A scan can be an abdominal scan, a pelvic scan or a scan of the femur, but must include the proximal femur at least as far as 20 mm below the lesser trochanter. One or more scans can be used to provide the necessary coverage of the bone and sufficient resolution. Preferably, pixel resolution is about 1-3 mm/slice in the axial (z) direction and about 0.5-1 mm/pixel in the x-y plane. For a typical CT scan, a kilovoltage peak (KVp) is about 120. Fully autonomous CT-based finite element analyses of a femur can be performed as schematically illustrated (1000) in FIG. 1. As shown in FIG. 1, at least one scan, preferably a quantitative CT (QCT) scan is performed (1100). The geometry of the femur (1200) is automatically segmented from the scan(s) (1300) and a geometrical representation of the femur automatically generated (1400). Inhomogeneous isotropic material properties are assigned to all points within the femur (1500) based on the Hounsfield Unit (HU) in the CT scan, as described hereinbelow, and the femur is meshed (1600), typically with a high-order tetrahedral mesh, resulting in a meshed femur with inhomogeneous isotropic material properties. Two loading configurations are applied (1700) and average maximum principal strains are extracted automatically (1800) over a circular region of about 10 mm² on the surface of the femur in each region of interest.

The femur's response under physiological loading can be well described by the linear theory of elasticity and, although the bone on the macroscopic level is orthotropic, excellent results can be obtained using isotropic inhomogeneous constitutive models for stance position loadings (see Yosibash et al. (2014)²² and Yosibash et al. (2007)²³ and references therein) and in sideway fall loading as presented in Altai et al.¹⁴ isotropic inhomogeneous constitutive models being models where, at each point within the femur, the mechanical response does not depend on the direction (the mechanical response in all directions is the same), but the mechanical response at different points differs; one region of the bone can be “stiffer” or “softer” than another region. Therefore, a linear elastic finite element analysis is performed. The numerical error is controlled by monitoring the error in energy norm and the maximum and minimum principal strains at the locations of interest as the polynomial degree of the elements is increased from 1 to 8.

### Material Properties Assigned to the FE Models

The relationships between Young's modulus and ash density for cortical²⁴ and trabecular bone²⁵, has been validated in experimental settings²⁶ and can be used to determine Young's modulus in the femur:

ρ_{K2HPO4}=10⁻³ (a×HU+b) [g/cm³] (1)

ρₐₛₕ=0.877×1.21×ρ_{K2HPO4}+0.08 [g/cm³] (2)

E_{cort}=10200×ρₐₛₕ^{2.01} [MPa], ρₐₛₕ≥0.486 [g/cm³] (3)

E_{trab}=2398 [MPa], 0.3<ρₐₛₕ<0.486 [g/cm³] (4)

E_{trab}=33900×ρₐₛₕ^{2.2} [MPa], ρₐₛₕ≤0.3 [g/cm³] (5)

Since most clinical CT scans are phantomless, a and b in eq. 1 are determined by the method applied in Sternheim et al.²⁷. The Poisson ratio was set to the constant value of v=0.3.

Boundary Conditions for Sideways Fall Position

In most cases of proximal femur fracture, the patient falls on the side hitting the floor with the lateral end of the greater trochanter. To simulate a sideways fall, the angles of the force on the femoral head must be defined properly and repeatably by determining anatomical landmarks (since each patient's position in the CT scanner is different) and assigning the location and direction of the applied forces based on patient-specific anatomical locations. In most of the prior art, little information is provided on the loading direction. Herein, three anatomical points are defined: the center of the femoral head (HC); the center of the shaft section center 20 mm distal to the lesser trochanter (SC); and the intercondylar notch (IN—the deep notch point between the rear surfaces of the medial and lateral epicondyles). In the case that only the proximal femur is scanned and the intercondylar notch is absent from the scanned image, the intercondylar point is estimated by a K Nearest Neighbor (KNN) algorithm so that automatic determination of the location of the intercondylar point can be independent of the fraction of the femur present in the scan. The KNN algorithm, a classification algorithm, determines the location of the intercondylar point from the outer surface of the femur as follows. A cloud of points on the outer surface of the femur is created for the proximal femur of interest. This is then compared to the cloud of points of the entire femur outer surface found in the training phase. The comparison of the cloud of points is performed by a cloud compare algorithm. The KNNs can be trained either manually or automatically on segmented scans of whole femurs. During training, scans comprising the intercondylar notch are provided and the location of the IN is identified in the scan. A portion of the scan is then removed so that only the proximal portion of the femur remains and the IN is no longer present in the scan. The location of the IN is redetermined and can be compared to the actual IN location. The step of removing a portion of the scan can be repeated.

Other algorithms that can be used in other embodiments for estimating the location of the intercondylar notch include Support Vector Machine (SVM) learning and Decision Tree Learning. AdaBoost can also be applied.

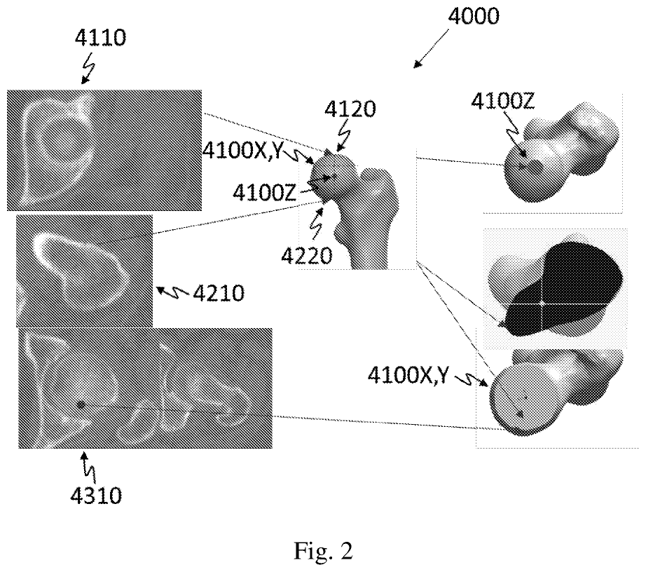

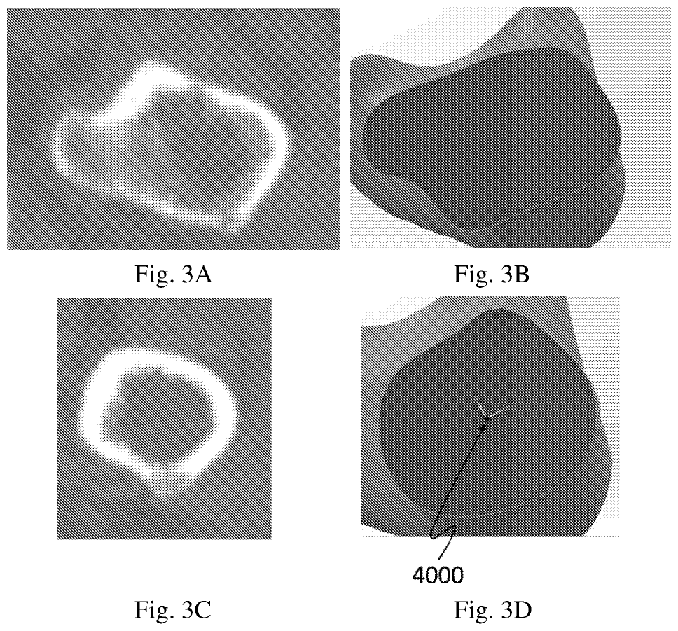

An algorithm to determine the head center (HC) is schematically illustrated in FIG. 2. First the most proximal point on the femoral head (in scan, 4110; in bone from front, 4120) and most distal one (in scan, 4210; in bone from front, 4220) are found and the sections (slices) of the scan between these two points are identified. The Z coordinate of the middle section between these sections, the section about halfway between the distalmost point and the proximalmost point, defines the Z coordinate of the HC (4100Z). The center of the section located 10 mm distal to the most proximal femoral head section (4100X,Y) defines the X, Y coordinates of the HC. The The location of the center of the shaft (SC) is schematically illustrated in FIG. 3: The lesser trochanter is identified in the CT scan (FIG. 3A in scan, FIG. 3B on bone) and a location 20 mm distal to the lesser trochanter is determined (FIG. 3C in scan, FIG. 3D on bone). The SC is the center of the shaft area (4000) at the location 20 mm distal to the lesser trochanter.

Once the three points HC, SC and IN are determined, the origin of the coordinate system is placed at the IN, with the axial axis defined by points IN and SC, so that angles γ and δ are determined:

The ‘frontal’ plane is determined by the three points HC, SC and IN. The shaft axis is then determined using two points (IN and SC). The γ angle is measured in the frontal plane from the shaft axis—along γ angle define the γ axis. The ‘perpendicular’ plane (P-P plane) is defined from the HC and a perpendicular to the γ axis as follows: In the P-P plane, create the δ axis, which passes through the HC and is perpendicular to the γ axis in the anterior detection (opposite to the lesser trochanter). The γ-angle is between the γ axis and the δ axis.

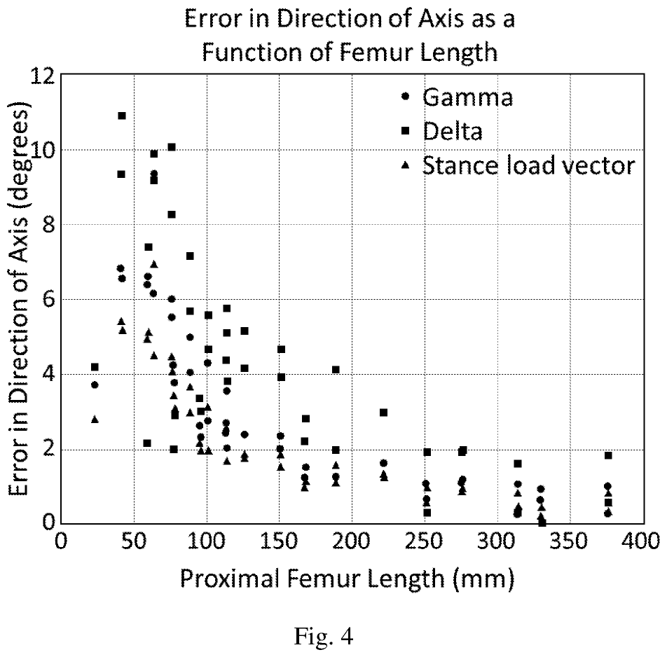

As an example, FIG. 4 shows the error in the angles γ and δ due to estimation of the intercondylar notch as a function of the proximal femur length remaining in the CT scan for three different femurs. In most the CT lower abdomen scans, the proximal femur's length is more than 150 mm so the error in the angles γ and δ is typically less than 4 degrees if the intercondylar notch is missing from the scan.

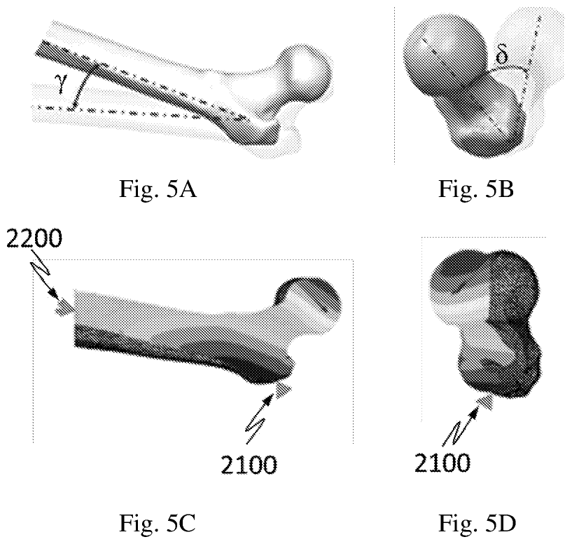

Based on the three anatomical points, the frontal plane of the femur, the shaft axis and the perpendicular to the frontal plane axis can be uniquely defined. According to Kazley et al.²⁸ the force at sideways fall is to be applied at specific angles: γ (FIG. 5A) and δ (FIG. 5B), where the angle γ represents the angle between the shaft and the ground during impact and is related to the amount of knee flexion present if the foot is on the ground, and δ reflects the amount of internal or external rotation of the femur relative to the ground. Typically, γ is in a range between 10° and 30°, and δ is in a range between 15° and 25° (See Kazley et al.²⁸ and references therein). Let m denote the vector along which the load is applied, then the displacement constraints on the greater trochanter (FIG. 5C, 5D, 2100) are: zero in the \vec{m} direction and traction free constraints in the plane perpendicular to \vec{m}. The distal shaft is constrained to zero displacement along the shaft axis (FIG. 4C, 2200).

It has been found that neck fractures are associated with one set of specific angles, (γ_{N}, δ_{N}), where γ_{N}=10±4° and δ_{N}=15±4°, while pertrochanteric fractures are associated with another set of specific angles, (γ_{P}, δ_{P}), where γ_{P}=30±4° and δ_{P}=45±4°. Table 1 shows a comparison between the angles as specified herein and the angles from the prior art.

TABLE 1
Loading angles

| | Gamma | Delta |
|---|---|---|
| Fall_{N} | 10 | 15 |
| Fall_{P} | 30 | 45 |
| Keyak²⁹ | 20 | 35 |
| Bessho³⁰ | 30 | 15 |
| van den Munckhof¹³ | 10-30 | 15-25 |

Bone Strength Computation

Typically, a fracture of the proximal femur due to a fall on the side is either a neck fracture (Fall_{N}) or a pertrochanteric fracture (Fall_{P}), with an almost equal probability of occurence^{31,32}. Thus, the fracture load can be estimated by an average of the load needed to induce neck fracture (Fall_{N} load) and the load to induce a pertrochanteric fracture (Fall_{P} load), with the fracture load being a load sufficient to induce a compressive strain equal to or greater than a fracture strain, as disclosed below. This average load can be normalized by the patient's body weight (BW) to obtain a body weight factor (BWF). For non-limiting example, a patient with a BWF of 2.1 requires 2.1 times the body weight to induce fracture and is at a higher risk of fracture than a patient with a BWF of 4.5.

For a femur of interest, an algorithm to compute a BWF using FEA can comprise: Perform at least one FEA with a Fall_{N} load and at least one FEA with a Fall_{P} load. For each FEA, the magnitude of the load is typically equal to the body weight (1.0BW), although other magnitudes can be used for the loading force.

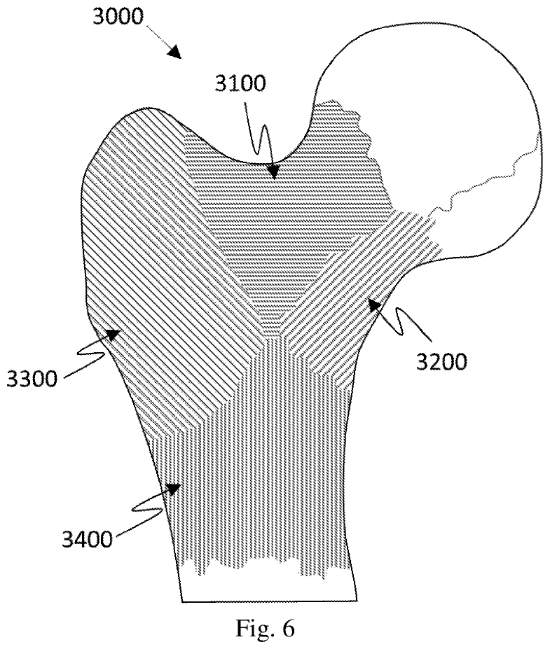

For each analysis, an averaged maximum tensile principal strain, E1ₘₐₓ, and an averaged maximum magnitude compressive principal strain, E3ₘᵢₙ, both averaged over a predefined area in a range between 3 mm² and 12 mm², preferably 10 mm², more preferably 5 mm², can be determined for each of four regions. As shown in FIG. 6, the four regions (3000) can be the anterior and posterior superior neck (3100), the anterior and posterior inferior neck (3200), the greater trochanter with the posterior lesser trochanter (3500+posterior of 3600), and the anterior lesser trochanter (anterior of 3600).

For each of the four regions, the results are compared, respectively, to the tensile yield strain, 7300 μstrain³³ and to the compressive yield strain, −8600 μstrain³⁴. Since an elastic linear analysis is performed, the determination of the BWF in tension (eq. 6) and compression (eq. 7) for each region is determined from:

$$B W {F}_{ten} = \frac{0.0073}{BW \cdot E {1}_{max}} \qquad ( 6 )$$

$$BW {F}_{com} = - \frac{0.0 \; 086}{BW \cdot E {3}_{min}} \qquad ( 7 )$$

As described above, FallN predicts a probability of fracture at the superior neck in compression and FallP predicts a probability of a pertrochanteric fracture, also in compression. Therefore, the average BWF for a femur, the hip strength score (HSS), can be calculated from:

$$H S S = B W {F}_{a v g} = \frac{1}{2} ( B W {F}_{com , FallN} + B W {F}_{com , FallP} ) \qquad ( 8 )$$

The HSS is preferably calculated for both femurs, with the lower HSS being used for subsequent statistical analysis.

From studies, such as retrospective studies, of patients with and without broken femurs, an HSS value can be found that is useful for differentiating between persons likely to break a femur and persons unlikely to break a femur. A non-limiting example of such a study is given hereinbelow, the example being for patients with T2DM and an exemplary differentiating HSS value of about 2.2 is found. The differentiating HSS value can depend on the type of medical condition, on the sex of the patient, on the age of the patient, on the patient's ethnicity, on comorbidity factors, on medication the patient is taking, on drugs the patient is using, on the patient's diet and any combination thereof. The effect of the patient's weight, as described above, is included in the calculation of the HSS. For T2DM, as shown hereinbelow, age, comorbidity factors and medication taken appear to have little effect on the differentiating HSS value.

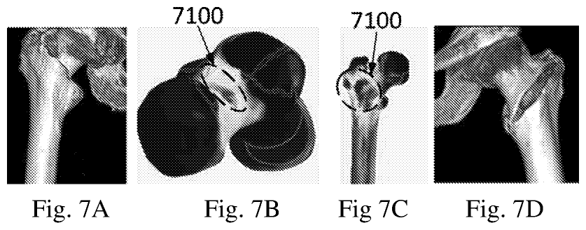

Illustrative examples of the two loading conditions and the maximum compressive strain locations are presented in FIG. 7A-D. FIG. 7A illustrates a neck fracture, with FIG. 7B showing the corresponding FEA principal strains from a Fall_{N} loading. FIG. 7D illustrates a pertrochanteric fracture, with FIG. 7C showing the corresponding FEA principal strains from a Fall_{P} loading. The areas of highest strain in FIG. 7B and FIG. 7C are indicated by the dashed circles (7100).

### Example 1—Preliminary Analysis for T2DM Patients

A retrospective cohort study demonstrated the use of an automated FEA analysis on existing abdomen or pelvis CT scans for assessing hip fracture risk in diabetic patients by comparing two groups of type 2 diabetic patients: patients in the first group, the study group, had a hip fracture within a year following a CT scan and patients in the second group, the control group, did not have a hip fracture within the year following a scan. The study was conducted at an urban tertiary medical center after IRB approval. The primary outcome was a difference in HSS between the study group and the control group. A secondary outcome was evaluation of the performance of HSS as a risk factor for sustaining hip fracture. Since no previous data were available regarding the distribution of HSS in the diabetic population, the sample size (number of patients in each group) was arbitrarily set at 30 patients in each group. A post-hoc power analysis determined that this study had a power of 88% to detect a difference in the HSS between the 2 groups.

For all members of both groups, the HSS was determined, as described above, from FEA analyses of CT scans.

Patient Population

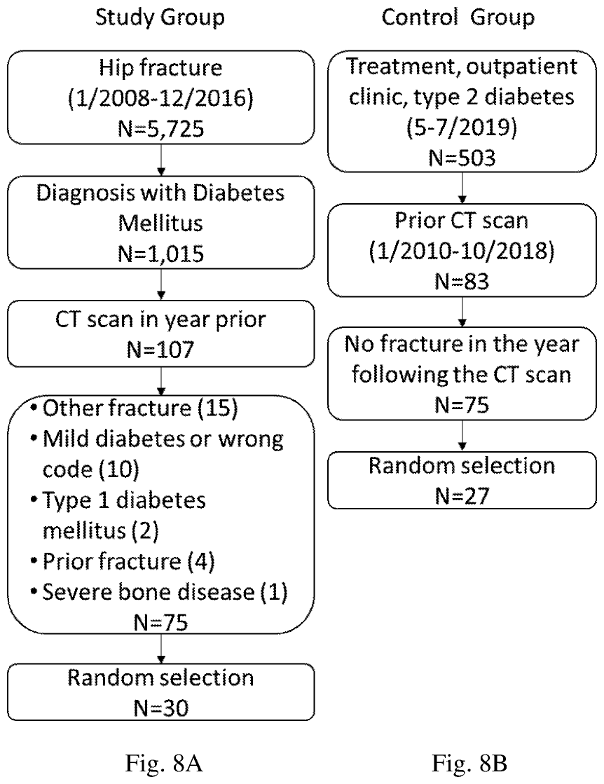

FIG. 8A-B shows the method of selection of patients for the study group (FIG. 8A) and the control group (FIG. 8B). As shown in FIG. 8A, the criteria for inclusion in the study group were: (1) A documented hip fracture between January 2008 and December 2016; (2) An abdomen or pelvis CT scan preformed within a year prior to the fracture including the proximal ⅓ of the femur; (3) Diagnosis of diabetes mellitus. A total of 113 patients met the inclusion criteria. Exclusion criteria of the study group included: (1) Another type of fracture, such as a pathologic fracture, subtrochanteric or atypical fracture, or a high energy fracture; (2) Mild diabetes controlled with diet change only or no diabetes (wrong coding); (3) Type 1 diabetes mellitus; (4) A prior fracture or prior orthopedic surgery on the same hip (re-fracture). 38 cases were excluded leaving 75 patients. Of these, 30 patients were selected arbitrarily for the study group.

As shown in FIG. 8B, the control group included 27 patients selected arbitrarily from the hospital's diabetes outpatient clinic who had had an abdomen or pelvis CT scan preformed between January 2010 and October 2018 and a documented 1-year follow-up after the CT scan showing no hip fracture. Exclusion criteria were the same as the study group.

The CT scans used were all abdominal or pelvic CT scans, with pixel resolution in the axial (z) direction of 1-3 mm/slice and 0.5-1 mm/pixel in the x-y plane, and a KVP of 120. Fully autonomous CT-base finite element analyses of all femurs were performed by the software product Simfini™ (PerSimiO Ltd, Beer-Sheva, Israel) according to the algorithm previously published^{26,27,35}, as schematically illustrated above (1000) in FIG. 1. The geometry of the femurs was automatically segmented from the Quantitative CT (QCT) scans. The resulting 3D segmented surface was auto-meshed by tetrahedral high-order elements. Inhomogeneous isotropic material properties were assigned to all points within the femur based on the Hounsfield Unit (HU) in the CT scan as detailed above. Two loading configurations were applied, as discussed above (FIGS. 5A-D) and average maximum principal strains were extracted automatically over a circular region of 10 mm² on the surface of the femur in each region of interest.

A statistical analysis was used to determine an HSS which can, with high probability, distinguish between a femur likely to break and a femur unlikely to break.

Based on multivariate analysis, the Hip Strength Score (HSS) is an independent factor associated with hip fractures after controlling for age, gender, BMI, smoking and diabetes treatment (Table 2). The optimal cut-off point for the HSS as a predictor of hip fracture was 2.2. Using this cut-off point, the multivariate logistic model showed sensitivity of 89%, specificity of 76% and an AUC of 0.90. According to this cut-off point, the odds of having a hip fracture were 17 times greater in patients who had HSS≤2.2 than in patients with HSS>2.2.

TABLE 2
Factors Tested for Association with Hip Fractures

| | Odds Ratio | | | P |
|---|---|---|---|---|
| | (95% Confidence | | P | Value < |
| Variable | Interval) | | value | 0.05? |
| Combined score | | | | |
| ≤2.2 | 17 | (1.9-63) | | — |
| >2.2 | Reference | | 0.012 | Yes |
| Age (per year) | 1.1 | (0.9-1.2) | 0.077 | No |
| Gender | | | | |
| Male | Reference | | | — |
| Female | 2.4 | (0.5-12) | 0.279 | No |
| BMI (per kg/m²) | 0.9 | (0.7-1.1) | 0.277 | No |
| Smoking | | | | |
| No | Reference | | | — |
| Yes | 3.3 | (0.5-19) | 0.194 | No |
| Diabetes treatment | | | | |
| Insulin + oral agent | Reference | | | — |
| Insulin alone | 5.0 | (0.7-26) | 0.112 | No |
| Oral agent alone | 3.6 | (0.5-13) | 0.186 | No |

As shown in Table 2, there were no statistically significant differences between the cases (broken femur) and the controls (no broken femur) for sex, BMI, time since diabetes diagnosis, diabetes treatment, osteoporosis treatment, history of malignancy, and history of metabolic bone disease or the use of mobility aids.

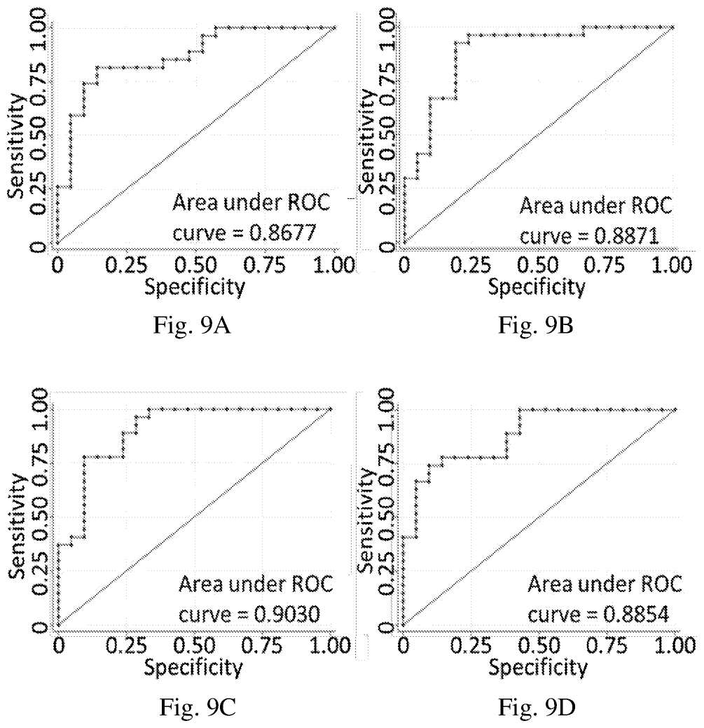

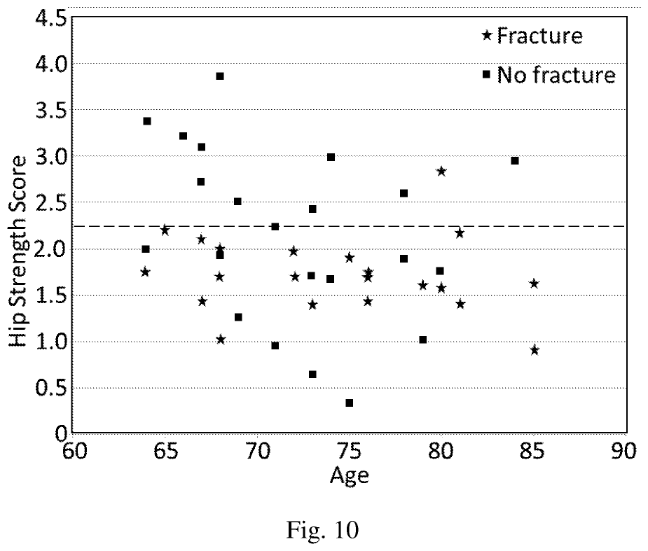

Further indication that an HSS of about 2.2 clearly differentiates between patients with a femur likely to fracture and patients with femurs unlikely to fracture is given by FIGS. 9 and 10. FIG. 9A-D shows ROC curves for the multivariate logistic regression model, with four different cut-off points for the combined score: FIG. 9A, cut-off point 1.3; FIG. 9B, cut-off point 1.8; FIG. 9C, cut-off point 2.2 and FIG. 9D, cut-off point 2.5. The ROC curve for a cut-off point of 2.2 (FIG. 9C) showed the best discriminating ability and the largest area under the ROC curve.

FIG. 10 shows the HSS for 27 patients with a hip fracture (stars) and 24 patients with no fracture (squares) as a function of age. It can be clearly seen that an HSS of about 2.2 (dashed line) differentiates between patients with a fracture and patients without a fracture. It can also be seen that only one of the patients with hip fracture had an HSS above 2.2, although almost half of the patients with no fracture had HSS below 2.2. Therefore, the HSS is more likely to generate false positives than false negatives, an outcome safer for the patients.

As shown in Table 3, the prevalence of diabetic complications was not significantly different between cases and controls with the exception of diabetic neuropathy. Cases with hip fractures were significantly older than controls (77 vs. 70 respectively, p=0.002), and had a significantly lower level of HbA1c (6.7 vs. 8.2 respectively, p=0.003).

TABLE 3
Patient characteristics and diabetes-related variables
for patients with and without fragility fractures

| | | | | | P |
|---|---|---|---|---|---|
| | | Fracture (%) | | P | Value < |
| | Total | Yes | No | value | 0.05? |
| No. patients | 51 | 27 | 24 | — | — |
| Age (yr) | 74 ± 8 | 77 ± 8 | 70 ± 6 | 0.002 | Yes |

Sex

| | | | | | | | |
|---|---|---|---|---|---|---|---|
| Male | 23 | 11 | (41) | 12 | (50) | 0.597 | No |
| Female | 28 | 16 | (59) | 12 | (50) | | |

| | | | | | |
|---|---|---|---|---|---|
| Body mass index | 27 ± 4.6 | 26 ± 4.4 | 28 ± 4.6 | 0.064 | No |
| (kg/m²) | | | | | |

Time since diabetes diagnosis

| | | | | | | | |
|---|---|---|---|---|---|---|---|
| <10 years | 6 | 1 | (4) | 5 | (23) | 0.201 | No |
| 10-20 years | 15 | 6 | (22) | 11 | (46) | | |
| >20 years | 12 | 8 | (30) | 6 | (25) | | |
| Unknown | 18 | 12 | (44) | 2 | (8) | | |

| | | | | | |
|---|---|---|---|---|---|
| Most recent HbA1c | 7.5 ± 1.6 | 6.7 ± 0.9 | 8.2 ± 1.8 | 0.003 | Yes |
| (%) | | | | | |
| Most recent | 1.3 ± 1.3 | 1.6 ± 1.7 | 1.0 ± 0.7 | 0.075 | No |
| creatinine (mg/dL) | | | | | |

Diabetes complications
Diabetic nephropathy

| | | | | | | | |
|---|---|---|---|---|---|---|---|
| Yes | 15 | 8 | (30) | 7 | (29) | 0.971 | No |
| No | 36 | 19 | (70) | 17 | (71) | | |

Diabetic neuropathy

| | | | | | | | |
|---|---|---|---|---|---|---|---|
| Yes | 10 | 2 | (7) | 8 | (33) | 0.033 | Yes |
| No | 41 | 25 | (93) | 16 | (67) | | |

Diabetic retinopathy

| | | | | | | | |
|---|---|---|---|---|---|---|---|
| Yes | 15 | 8 | (30) | 7 | (29) | 0.971 | No |
| No | 36 | 19 | (70) | 17 | (71) | | |

Peripheral vascular disease

| | | | | | | | |
|---|---|---|---|---|---|---|---|
| Yes | 6 | 4 | (15) | 2 | (8) | 0.671 | No |
| No | 45 | 23 | (85) | 22 | (92) | | |

Diabetic foot

| | | | | | | | |
|---|---|---|---|---|---|---|---|
| Yes | 3 | 1 | (4) | 2 | (8) | 0.595 | No |
| No | 48 | 26 | (96) | 22 | (92) | | |

Diabetes treatment

| | | | | | | | |
|---|---|---|---|---|---|---|---|
| Insulin alone | 14 | 9 | (33) | 5 | (21) | 0.061 | No |
| Oral agents alone | 22 | 14 | (52) | 8 | (33) | | |
| (sulfonylurea, non- | | | | | | | |
| sulfonylurea) | | | | | | | |
| Insulin added to | 15 | 4 | (15) | 11 | (46) | | |
| an oral agent | | | | | | | |
| (sulfonylurea, non- | | | | | | | |
| sulfonylurea) | | | | | | | |

Osteoporosis treatment

| | | | | | | | |
|---|---|---|---|---|---|---|---|
| Calcium + | 15 | 7 | (27) | 8 | (33) | 0.876 | No |
| Vitamin D | | | | | | | |
| Bisphosphonates | 1 | 1 | (4) | 0 | (0) | | |
| None | 34 | 18 | (69) | 16 | (67) | | |

History of malignancy

| | | | | | | | |
|---|---|---|---|---|---|---|---|
| Yes | 20 | 12 | (44) | 8 | (33) | 0.567 | No |
| No | 31 | 15 | (56) | 16 | (67) | | |

History of metabolic bone disease

| | | | | | | | |
|---|---|---|---|---|---|---|---|
| Yes | 2 | 1 | (4) | 1 | (4) | 1.00 | No |
| No | 49 | 26 | (96) | 23 | (96) | | |

Use of mobility aids

| | | | | | | | |
|---|---|---|---|---|---|---|---|
| Yes | 20 | 14 | (52) | 6 | (26) | 0.064 | No |
| No | 30 | 13 | (48) | 17 | (74) | | |

For this preliminary study, in both the study group and the control group, three patients were excluded since the CT scan was insufficient to calculate the HSS (the scan did not include the proximal femur to the lesser trochanter), putting the CT utilization rate at 89%. Thus, the analysis included 27 cases in the fracture group and 24 cases in the control group.

The hip strength score (HSS) was significantly lower in the cases with hip fracture (1.76, SD 0.46) than in the controls (2.31, SD 0.74) (p=0.002).

Therefore, the HSS can assess hip fracture risk in a population of T2DM patients.

In some populations of patients, a cut-off point for the HSS as a predictor of hip fracture can be in a range from 1.5 to 4.5.

In some populations of patients, a cut-off point for the HSS as a predictor of hip fracture can be in a range from 2.0 to 2.3.

### REFERENCES

1. Hernlund E, Svedbom A, Ivergard M, et al. Osteoporosis in the European Union: medical management, epidemiology and economic burden. A report prepared in collaboration with the International Osteoporosis Foundation (IOF) and the European Federation of Pharmaceutical Industry Associations (EFPIA). Arch Osteoporos 2013; 8: 136. 2013/10/12. DOI: 10.1007/s11657-013-0136-1.
2. Kanis J A, Oden A, McCloskey E V, et al. A systematic review of hip fracture incidence and probability of fracture worldwide. Osteoporos Int 2012; 23: 2239-2256. 2012/03/16. DOI: 10.1007/s00198-012-1964-3.
3. Shaw J E, Sicree R A and Zimmet P Z. Global estimates of the prevalence of diabetes for 2010 and 2030. Diabetes Res Clin Pract 2010; 87: 4-14. 2009/11/10. DOI: 10.1016/j.diabres.2009.10.007.
4. Evidence-based clinical practice guideline: management of hip fracture in the elderly. 2014. AAOS.
5. Fan Y, Wei F, Lang Y, et al. Diabetes mellitus and risk of hip fractures: a meta-analysis. Osteoporos Int 2016; 27: 219-228. 2015/08/13. DOI: 10.1007/s00198-015-3279-7.
6. Johnell O, Kanis J A, Oden A, et al. Predictive value of BMD for hip and other fractures. J Bone Miner Res 2005; 20: 1185-1194. 2005/06/09. DOI: 10.1359/JBMR.050304.
7. Botella Martinez S, Varo Cenarruzabeitia N, Escalada San Martin J, et al. The diabetic paradox: Bone mineral density and fracture in type 2 diabetes. Endocrinol Nutr 2016; 63: 495-501. 2016/08/03. DOI: 10.1016/j.endonu.2016.06.004.
8. Giangregorio L M, Leslie W D, Lix L M, et al. FRAX underestimates fracture risk in patients with diabetes. J Bone Miner Res 2012; 27: 301-308. 2011/11/05. DOI: 10.1002/jbmr.556.
9. Schacter G I and Leslie W D. DXA-Based Measurements in Diabetes: Can They Predict Fracture Risk? Calcif Tissue Int 2017; 100: 150-164. 2016/09/07. DOI: 10.1007/s00223-016-0191-x.
10. Ferrari S L, Abrahamsen B, Napoli N, et al. Diagnosis and management of bone fragility in diabetes: an emerging challenge. Osteoporos Int 2018; 29: 2585-2596. 2018/08/02. DOI: 10.1007/s00198-018-4650-2.
11. Rubio J B, Barquero L D, Martinez J M O, et al. Review of the scientific evidence regarding clinical use of the Trabecular Bone Score (TBS) SEIOMM official position (2018). Rev Osteopor Metab M 2018; 10: 149-159. DOI: 10.4321/51889-836x2018000400008.
12. Johannesdottir F, Allaire B and Bouxsein M L. Fracture Prediction by Computed Tomography and Finite Element Analysis: Current and Future Perspectives. Curr Osteoporos Rep 2018; 16: 411-422. 2018/05/31. DOI: 10.1007/s11914-018-0450-z.
13. van den Munckhof S and Zadpoor A A. How accurately can we predict the fracture load of the proximal femur using finite element models? Clin Biomech (Bristol, Avon) 2014; 29: 373-380. 2014/02/04. DOI: 10.1016/j.clinbiomech.2013.12.018.
14. Altai Z, Qasim M, Li X S, et al. The effect of boundary and loading conditions on patient classification using finite element predicted risk of fracture. Clin Biomech 2019; 68: 137-143.
15. Lenchik L, Weaver A A, Ward R J, et al. Opportunistic Screening for Osteoporosis Using Computed Tomography: State of the Art and Argument for Paradigm Shift. Curr Rheumatol Rep 2018; 20: 74. 2018/10/15. DOI: 10.1007/s11926-018-0784-7.
16. Lee S J, Anderson P A and Pickhardt P J. Predicting Future Hip Fractures on Routine Abdominal CT Using Opportunistic Osteoporosis Screening Measures: A Matched Case-Control Study. AJR Am J Roentgenol 2017; 209: 395-402. 2017/06/02. DOI: 10.2214/AJR.17.17820.
17. Dagan N, Elnekave E, Barda N, et al. Automated opportunistic osteoporotic fracture risk assessment using computed tomography scans to aid in FRAX underutilization. Nat Med 2020; 26: 77-82. 2020/01/15. DOI: 10.1038/s41591-019-0720-z.
18. Kopperdahl D L, Aspelund T, Hoffmann P F, et al. Assessment of incident spine and hip fractures in women and men using finite element analysis of CT scans. J Bone Miner Res 2014; 29: 570-580.2013/08/21. DOI: 10.1002/jbmr.2069.
19. Orwoll E S, Marshall L M, Nielson C M, et al. Finite element analysis of the proximal femur and hip fracture risk in older men. J Bone Miner Res 2009; 24: 475-483. 2008/12/04. DOI: 10.1359/jbmr.081201.
20. Karim L, Rezaee T and Vaidya R. The Effect of Type 2 Diabetes on Bone Biomechanics. Curr Osteoporos Rep 2019; 17: 291-300. 2019/08/09. DOI: 10.1007/s11914-019-00526-w.
21. Adams A L, Fischer H, Kopperdahl D L, et al. Osteoporosis and Hip Fracture Risk from Routine Computed Tomography Scans: The Fracture, Osteoporosis, and CT Utilization Study (FOCUS). J Bone Miner Res 2018; 33: 1291-1301. 2018/04/18. DOI: 10.1002/jbmr.3423.
22. Yosibash Z, Plitman Mayo R, Dahan G, et al. Predicting the stiffness and strength of human femurs with real metastatic tumors. Bone 2014; 69: 180-190. 2014/10/07. DOI: 10.1016/j.bone.2014.09.022.
23. Yosibash Z, Trabelsi N and Milgrom C. Reliable simulations of the human proximal femur by high-order finite element analysis validated by experimental observations. J Biomech 2007; 40: 3688-3699. DOI: 10.1016/j.jbiomech.2007.06.017.
24. Keller T S. Predicting the Compressive Mechanical-Behavior of Bone. J Biomech 1994; 27: 1159-1168. DOI: Doi 10.1016/0021-9290(94)90056-6.
25. Keyak J H, Fourkas M G, Meagher J M, et al. Validation of an Automated-Method of 3-Dimensional Finite-Element Modeling of Bone. J Biomed Eng 1993; 15: 505-509.
26. Yosibash Z, Trabelsi N and Milgrom C. Reliable simulations of the human proximal femur by high-order finite element analysis validated by experimental observations. J Biomech 2007; 40: 3688-3699. DOI: 10.1016/j.jbiomech.2007.06.017.
27. Sternheim A, Giladi O, Gortzak Y, et al. Pathological fracture risk assessment in patients with femoral metastases using CT-based finite element methods. A retrospective clinical study. Bone 2018; 110: 215-220. 2018/02/24. DOI: 10.1016/j.bone.2018.02.011.
28. Kazley J M, Banerjee S, Abousayed M M, et al. Classifications in Brief: Garden Classification of Femoral Neck Fractures. Clin Orthop Relat R 2018; 476: 441-445. DOI: 10.1007/s11999.0000000000000066.
29. J. H. Keyak, H. B. Skinner and J. A. Fleming. Effect of force direction on femoral fracture load for two types of loading conditions. Journal of Orthopaedic Research 2001; 19: 539-544.
30. Bessho, M., Ohnishi, I., Okazaki, H. et al. Prediction of the strength and fracture location of the femoral neck by C T-based finite-element method: a preliminary study on patients with hip fracture. J Orthop Sci 9, 545-550 (2004).
31. Filipov O. Epidemiology and social burden of the femoral neck fractures. Journal of IMAB—Annual Proceedings (Scientific Papers) 2014; 20: 516-518. DOI: dx.doi.org/10.5272/jimab.2014204.516.
32. Mokawem M, Bobak P and Aderinto J. The management of pertrochanteric fractures of the hip. Orthopaedics and Trauma 2012; 26: 112-123.
33. Bayraktar H H, Morgan E F, Niebur G L, et al. Comparison of the elastic and yield properties of human femoral trabecular and cortical bone tissue. J Biomech 2004; 37: 27-35.
34. Dahan G, Trabelsi N, Safran O, et al. Finite element analyses for predicting anatomical neck fractures in the proximal humerus. Clin Biomech 2019; 68: 114-121.
35. Yosibash Z, Kent M, Trabelsi N, et al. Autonomous FEs (AFE)—A Stride Toward Personalized Medicine. Computers and Mathematics with Applications 2020; In Press.

1.-74. (canceled)

75. A method for determining at least three anatomical points of a femur from at least one clinical image comprising steps of:
segmenting said at least one clinical image to obtain a 3D image of said femur;
applying a location-determining algorithm selected from a group consisting of K Nearest Neighbor (KNN), Support Vector Machine (SVM) learning, Decision Tree Learning, and any combination thereof to determine a 3D location of an intercondylar notch for said 3D image;
determining a 3D location of a femur head center (HC) and a point along a shaft center (SC);
thereby determining said at least three anatomical points.

76. The method of claim 75, wherein said method comprises training said location-determining algorithm by steps of:
obtaining, for at least one femur, at least one scan of said femur, said scan including all of said at least three anatomical points;
determining, for at least one of said at least three anatomical points, a location, each said location being a determined anatomical point;
removing a portion from said 3D image to generate a training image, said removed portion comprising said at least one of said at least three anatomical points;
training said KNN by, for each of said at least one femurs, said KNN executing steps of:
determining, for at least one of said at least three anatomical points, a location, each said location being a training anatomical point;
generating, by comparing each of said training anatomical points and a corresponding determined anatomical point, an anatomical point difference factor;
comparing each said anatomical point difference factor with a range of a predetermined difference factor;
said anatomical point difference factor being outside said range of said predetermined difference factor, modifying said KNN; and
repeating said steps of determining, generating, comparing and modifying until each of said anatomical point difference factors being inside said range of said predetermined difference factor;

77. The method of claim 75, wherein said at least three anatomical points comprises a center of a femoral head; a center of a femoral shaft section a predetermined distance distal to the lesser trochanter; and a femoral intercondylar notch;
a. said step of determining the 3D location of the HC comprises sub-steps of:
identifying a most proximal point on the femur head;
identifying a most distal point on the femur head;
identifying a middle section of the femur head, said middle section being a section halfway between said most proximal point and said most distal point, a Z coordinate of said HC being a Z coordinate of said middle section;
selecting an HC section, said HC section being 10 mm distal to the most proximal point; and
determining a center of said HC section, an X coordinate of said HC being an X coordinate of said HC section and a Y coordinate of said HC being a Y coordinate of said HC section;

78. The method of claim 75, wherein said step of determining the 3D location of the SC comprises steps of:
identifying a location of a lesser trochanter in said scan;
selecting an SC section, said SC section being a predetermined distance distal to said lesser trochanter, a Z coordinate of said SC being a Z coordinate of said SC section; and
determining a center of said SC section, an X coordinate of said SC being an X coordinate of said SC section and a Y coordinate of said SC being a Y coordinate of said SC section

79. The method of claim 75, wherein said predetermined distance distal to said lesser trochanter is in a range from 10 mm to 30 mm; and

80. The method of claim 75, wherein said predetermined distance distal to said lesser trochanter is 20 mm.

81. The method of claim 76, wherein said range of said predetermined difference factor is different for at least two of said at least three anatomical points.

82. The method of claim 76, wherein at least one of the following is true:
a. said range of said predetermined difference factor is different for at least two of said at least three anatomical points;
b. said at least one of said at least three anatomical points is an intercondylar point;
c. said training image comprises a proximal portion of the femur;
d. said removed portion comprises said intercondylar point;
e. said predetermined distance distal to the lesser trochanter is 20 mm distal to the lesser trochanter; and
f. said predetermined distance distal to the lesser trochanter is 20 mm distal to the lesser trochanter.

83. A method of automatically generating bone-specific constraints for an FEA model of
a femur in an FEA coordinate system, comprising steps of:
defining a frontal plane in said FEA coordinate system from three predetermined anatomic points of said femur;
defining a perpendicular to said frontal plane;
defining a bone shaft axis from a predetermined two of said three predetermined anatomic points;
generating an angle γ, said angle γ being an angle between a force vector and said shaft axis;
generating an angle δ, the angle δ being an angle between said force vector and said perpendicular to said frontal plane;
applying a force vector with magnitude equal to a predetermined force value at said angles γ and δ;
applying zero displacement in a direction parallel to said force vector on a greater trochanter lateral end;
applying, at a distal end of said FEA model of said femur, a zero displacement in a direction parallel to said bone shaft axis and a zero displacement in a direction parallel to said perpendicular; and
applying, at a distal end of said FEA model of said femur, zero force in a direction perpendicular to both said shaft axis and said perpendicular.

84. The method of claim 83, wherein at least two sets of said bone specific constraints are generated, one of said at least two sets having differing from at least one other of said at least two sets in a member of a group consisting of angle γ, angle δ and any combination thereof.

85. The method of claim 83, wherein said predetermined force value is equal to a body mass of a patient with said femur.

86. The method of claim 83, wherein said three anatomical points are a center of a femoral head; a center of a femoral shaft section a predetermined distance distal to a lesser trochanter; and a femoral intercondylar notch.

87. The method of claim 83, wherein at least one of the following is true:
a. said at least two sets of said bone specific constraints comprises a set with angle γ in a range of 6° to 14° and angle δ in a range of 11° to 19°;
b. said at least two sets of said bone specific constraints comprises a set with angle γ in a range of 26° to 34° and angle δ in a range of 41° to 49°;
c. said at least two sets of said bone specific constraints comprises a set with angle γ being 10° and angle δ being 15°; and
d. said at least two sets of said bone specific constraints comprises a set with angle γ being 30° and angle δ being 45°.

88. A method of determining average strain in a femur from an FEA analysis of a femur under a predetermined load, comprising steps of:
automatically dividing a proximal end of said femur into four regions determined by anatomical points, said anatomical points being anterior and posterior superior neck, anterior and posterior inferior neck; greater trochanter and posterior lesser trochanter and anterior lesser trochanter; for each point on an exterior surface of said femur, averaging a principal compressive strain and a principal tensile strain over a predetermined area, generating an averaged principal compressive strain and an averaged principal tensile strain;
for each of said four regions, determining a maximum principal compressive strain, said maximum principal compressive strain being said averaged principal compressive strain having a maximum absolute value of said nodal averaged principal compressive strain of said area in said region; and
for each of said four regions, determining a maximum principal tensile strain, said maximum principal tensile strain being a maximum value of said averaged principal tensile strain of said region.

89. The method of claim 88, wherein said four regions are a superior neck, an inferior neck, an anterior trochanter and a posterior trochanter;

90. The method of claim 88, wherein said predetermined area is in a range from 2.5 mm² to 10 mm²;

91. The method of claim 88, wherein said predetermined area is 5 mm².

92. A method of determining likelihood of fracture of a femur, comprising steps of:
executing at least two FEA analyses, said FEA analyses having constraint sets FallN and FallP;
extracting a maximum tensile principal strain E1max for said femur;
extracting a maximum compressive principal strain E3 min for said femur;
for each of said four regions and for each of said constraint sets, calculating a body weight factor in tension (BWFten) for each said region from
$$B W {F}_{t e n} = \frac{0.0073}{BW \cdot E {1}_{max}}$$
where BW is the body mass of a patient with said femur and E1max is said maximum tensile principal strain;
for each of said four regions and for each of said constraint sets, calculating a body weight factor in compression (BWFcom) for each said region from
$$B W {F}_{c o m} = - \frac{0.0086}{BW \cdot E {3}_{min}}$$
where BW is the body mass of a patient with said femur and E3 min is said maximum compressive principal strain;
for each of said four regions, Use BWF_{com,FallN} and/or BWF_{com,FallP} where BWF_{com}, fallN for each region is the body weight factor in compression for that region under the constraint set FallN and where BWFcom,fallP for each region is the body weight factor in compression for that region under the constraint set FallP to determine risk of fracture.

93. The method of claim 92, wherein said constraint sets FallN and FallP have constraints comprising:
applying a force vector with magnitude equal to a predetermined force value at angles γ and δ;
applying zero displacement in a direction parallel to said force vector on a greater trochanter lateral end;
applying, at a distal end of said FEA model of said femur, a zero displacement in a direction parallel to a bone shaft axis and a zero displacement in a direction parallel to a perpendicular to a bone frontal plane;
applying, at a distal end of said FEA model of said femur, zero force in a direction perpendicular to both said bone shaft axis and said perpendicular to a bone frontal plane.

94. The method of claim 92, wherein at least one of the following is true:
a. said constraint set FallN comprises a set with angle γ in a range of 6° to 14° and angle δ in a range of 11° to 19°
b. said constraint set Fall_{P} comprises a set with angle γ in a range of 26° to 34° and angle δ in a range of 41° to 49°;
c. said constraint set FallN comprises a set with angle γ being 10° and angle δ being 15′;
d. said constraint set Fall_{P} comprises a set with angle γ being 30° and angle δ being 45°;
e. said predetermined force value is equal to a body mass of a patient with said femur;
